(19) United States
(12) Patent Application Publication
(10) Pub. No.: US 2026/0076736 A1
(43) Pub. Date: Mar. 19, 2026

(54) BALLOON CATHETER HAVING ABLATION AND RETURN ELECTRODES

(71) Applicant: Biosense Webster (Israel) Ltd., Yokneam (IL)

(72) Inventors: Assaf GOVARI, Haifa (IL); Christopher Thomas BEECKLER, Brea, CA (US); Joseph Thomas KEYES, Sierra Madre, CA (US); Kevin Justin HERRERA, West Covina, CA (US)

(73) Assignee: Biosense Webster (Israel) Ltd., Yokneam (IL)

(21) Appl. No.: 19/397,579

(22) Filed: Nov. 21, 2025

Related U.S. Application Data
(63) Continuation of application No. 16/994,428, filed on Aug. 14, 2020.

(51) Int. Cl. A61B 18/14; A61B 18/00; A61B 18/12

(52) U.S. Cl. CPC A61B 18/1492; A61B 18/1206; A61B 2018/0022; A61B 2018/00267; A61B 2018/00577; A61B 2018/1253

(57) ABSTRACT

A catheter includes an expandable frame for insertion into an organ of a patient, one or more first electrodes, and a second electrode. The one or more first electrodes are disposed on the expandable frame at one or more first positions for placing in contact with a target tissue of the organ, and are configured to perform one or both of: (i) sensing one or more electrical signals from the target tissue, and (ii) applying one or more ablation pulses to the target tissue. The second electrode is disposed within an internal volume of the expandable frame, at a second position that is not in contact with the target tissue while the one or more first electrodes contact the target tissue, and is configured to serve as a return or common electrode for the electrical signals.

### CROSS-REFERENCE TO RELATED APPLICATIONS

This application is a continuation of prior filed U.S. patent application Ser. No. 16/994,428 filed Aug. 14, 2020 (Attorney Ref. No.: BIO6372USNP1-253757.000289). The entire contents of which are hereby incorporated by reference.

### FIELD OF THE INVENTION

The present invention relates generally to medical devices, and particularly to methods and systems for performing unipolar ablation procedures using a catheter having an expandable frame.

### BACKGROUND OF THE INVENTION

Various types of diagnostic and therapeutic catheters, such as balloon catheters, may be used in mapping and/or treatment applications, such as in unipolar ablation of a patient organ.

For example, U.S. Patent Application Publication 2016/0199127 describes tools and methodologies for treating systemic nerve hyperactivity through splenic and/or carotid denervation. The invention discloses devices for performing ablation and protecting a patient from formation of embolisms, and well as an ablation unit for performing branching ablation.

U.S. Pat. No. 9,925,001 describes a renal nerve ablation device including an elongate tubular member having a distal region. An expandable member may be coupled to the distal region. One or more active electrodes may be coupled to the expandable member. One or more ground electrodes may be coupled to the expandable member. The one or more active electrodes and/or the one or more ground electrodes may be oriented helically about the length of the expandable member.

### SUMMARY OF THE INVENTION

An embodiment of the present invention that is described herein provides a catheter including an expandable frame for insertion into an organ of a patient, one or more first electrodes, and a second electrode. The one or more first electrodes are disposed on the expandable frame at one or more first positions for placing in contact with a target tissue of the organ, and are configured to perform one or both of: (i) sensing one or more electrical signals from the target tissue, and (ii) applying one or more ablation pulses to the target tissue. The second electrode is disposed within an internal volume of the expandable frame, at a second position that is not in contact with the target tissue while the one or more first electrodes contact the target tissue, and is configured to serve as a return or common electrode for the electrical signals.

In some embodiments, the catheter includes an ablation power source, which is electrically connected to the catheter and is configured to apply one or more unipolar ablation pulses to the one or more first electrodes. In other embodiments, the organ includes a patient heart, and the one or more electrical signals include one or more unipolar intra-cardiac electrical signals sensed from the heart. In yet other embodiments, the organ includes a patient heart, and the one or more ablation pulses include one or more unipolar radiofrequency (RF) ablation pulses applied to the heart.

In an embodiment, the expandable frame includes an inflatable balloon. In another embodiment, the expandable frame includes an expandable basket.

There is additionally provided, in accordance with an embodiment of the present invention, a method for producing a catheter, the method including receiving an expandable frame for insertion into an organ of a patient. One or more first electrodes for placing in contact with a target tissue of the organ are disposed on the expandable frame, at one or more first positions. A second electrode, which serves as a return or common electrode for the electrical signals, is disposed within an internal volume of the expandable frame, at a second position that is not in contact with the target tissue while the one or more first electrodes contact the target tissue.

In some embodiments, disposing the one or more first electrodes includes producing the one or more first electrodes in the expandable frame. In other embodiments, disposing the second electrode includes coupling the second electrode to a catheter shaft at the second position, which is within the internal volume of the expandable frame.

In an embodiment, the method includes disposing at a third position that is not in contact with the target tissue while the one or more first electrodes contact the target tissue, a third electrode that serves as an additional return electrode for the electrical signals. In another embodiment, disposing the third electrode at the third position includes disposing the third electrode on a catheter shaft out of the internal volume of the expandable frame.

The present invention will be more fully understood from the following detailed description of the embodiments thereof, taken together with the drawings in which:

### BRIEF DESCRIPTION OF THE DRAWINGS

FIG. 1 is a schematic, pictorial illustration of a catheter-based position-tracking and radiofrequency (RF) ablation system, in accordance with an embodiment of the present invention;

FIG. 2 is a schematic, pictorial illustration of a catheter tip of the RF ablation system, in accordance with an embodiment of the invention; and

FIG. 3 is a flow chart that schematically illustrates a method for producing a catheter tip comprising a balloon and multiple electrodes, in accordance with an embodiment of the invention.

### DETAILED DESCRIPTION OF EMBODIMENTS

### Overview

Unipolar, also referred to as monopolar, radiofrequency (RF) ablation procedures are used in several medical applications, such as in sensing and ablation of a patient heart. In principle, a physician may acquire the unipolar signals and/or apply the unipolar pulses using one or more sensing/ablation electrodes together with a return electrode patch that may be coupled to the external patient skin. In this configuration, however, the large distance between the sensing/ablation electrode(s) in contact with the target tissue of patient heart, and the return electrode may (i) add noise to the unipolar signals acquired from the tissue, and/or (ii) have an increased impedance to the unipolar signals applied to the tissue during the RF ablation procedure.

An embodiment of the present invention that is described herein provides an ablation system having a catheter comprising an expandable frame, such as an expandable balloon or an expandable basket, coupled to a catheter tip at the catheter distal end, which is inserted into a patient heart.

In some embodiments, the catheter comprises one or more sensing and/or ablation electrodes, which are disposed on the expandable frame at one or more positions for placing in contact with a target tissue (intended to be ablated) of the heart, and are configured to perform one or both of: (i) sensing one or more intra-cardiac electrical signals from the target tissue, and (ii) applying one or more RF ablation pulses to the target tissue.

In some embodiments, the catheter comprises an additional electrode, which is disposed within an internal volume of the expandable frame, at a position that is not in contact with the target tissue while the one or more sensing and/or ablation electrodes contact the target tissue. In such embodiments, the additional electrode is configured to serve as a return or common electrode for the electrical signals or the applied pulses, so as to: (i) enable acquisition of unipolar intra-cardiac electrical signals from the heart, and/or (ii) apply, using the ablation electrode(s), unipolar RF ablation pulses to the target tissue.

In some embodiments, the ablation system comprises a pulse generator, which is electrically connected to the catheter and is configured to apply the RF ablation pulses to the one or more ablation electrodes. The ablation system further comprises a processor, which is configured to receive the acquired intra-cardiac signals acquired by the sensing electrodes and to control the pulse generator to apply the RF ablation pulses to the ablation electrodes.

In some embodiments, the disclosed techniques may be used, mutatis mutandis, in other applications, such as in sensing and ablation procedures of renal nerves or sensing and/or ablation of other organs of a patient.

The disclosed techniques, and in particular the proximity between the sensing/ablation electrode and the return electrode that are disposed on the same catheter, reduce the noise level in electrical signals acquired from patient tissue, and improve accuracy of tissue ablation by having reduced impedance between the ablation electrode(s) and the return electrode used in the ablation procedure.

### System Description

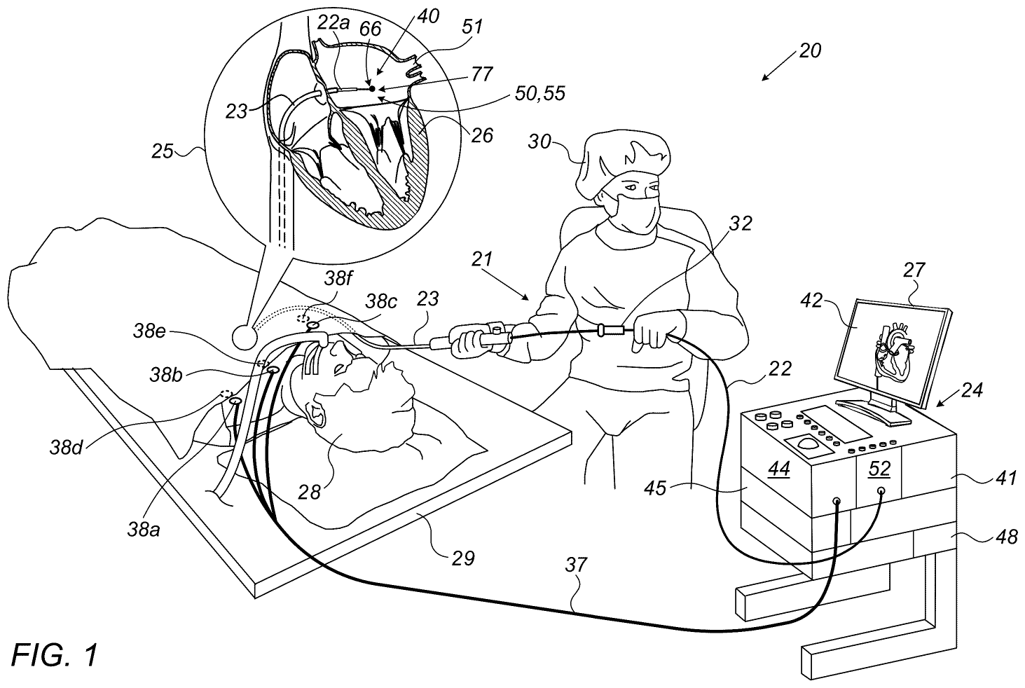

FIG. 1 is a schematic, pictorial illustration of a catheter-based position-tracking and radiofrequency (RF) ablation system 20, in accordance with an embodiment of the present invention.

Reference is now made to an inset 25. In some embodiments, system 20 comprises a catheter tip 40 that is fitted at a distal end 22a of a shaft 22 of a catheter 21 shown in the general view of FIG. 1.

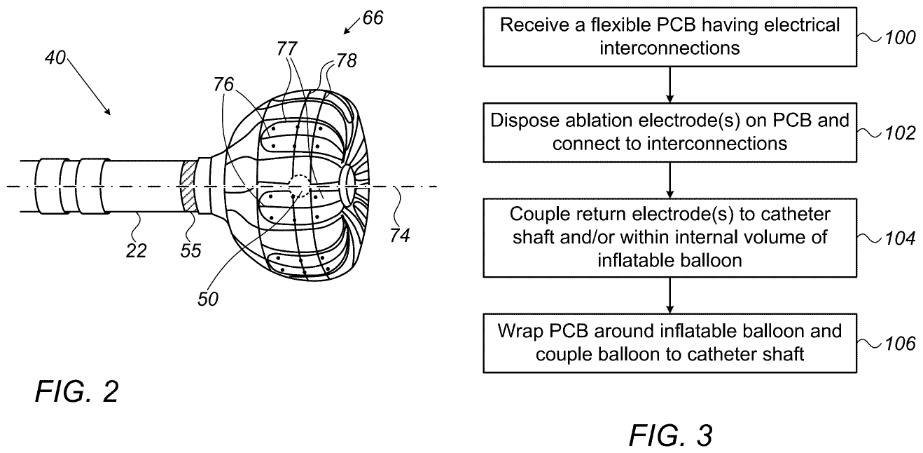

In some embodiments, catheter tip 40 comprises an expandable frame, in the present example, an inflatable balloon 66 having multiple electrodes, such as but not limited to (i) multiple sensing and/or RF ablation electrodes 77, and (ii) one or more electrodes 50 and 55, configured to serve as a return or common electrode as will be described in detail below. In some embodiments, electrodes 77 and electrodes 50 and 55 are used as return electrodes for acquiring unipolar signals from an ostium 51 of a pulmonary vein (PV) in a heart 26, or for ablating one or more unipolar ablation pulses to ostium 51 in heart 26. Balloon 66 and electrodes 50, 55 and 77 are described in detail in FIG. 2 below.

Reference is now made back to the general view of FIG. 1. In some embodiments, the proximal end of catheter 21 is connected to a control console 24 comprising an RF generator 45 for applying the unipolar ablation pulses to tissue of ostium 51. An ablation protocol comprising ablation parameters is stored in a memory 48 of console 24.

In some embodiments, a physician 30 inserts distal end 22a of shaft 22 through a sheath 23 into heart 26 of a patient 28 lying on a table 29. Physician 30 advances the distal end of shaft 22 to ostium 51, also referred to herein as a target location, in heart 26 by manipulating shaft 22 using a manipulator 32 near the proximal end of catheter 21. During the insertion of distal end 22a, catheter tip 40 is maintained inside sheath 23 so as to minimize vascular trauma along the way to target location.

In some embodiments, system 20 comprises an impedance-based active current location (ACL) system, which may be used by physician 30 for navigating and tracking the position of catheter tip 30 in heart 26.

In an embodiment, physician 30 navigates the distal-end of shaft 22 to the target location by tracking the position of catheter tip 40. During navigation of distal end 22a in heart 26, console 24 receives signals from a coil (not shown) or from any other element, e.g., any of electrodes 50 and 55, which is configured to serve as an impedance-based position sensor of the ACL system.

In some embodiments, the ACL system comprises a plurality of electrodes 38, which are coupled to the body of patient 28, e.g., via patches 29 that adhere to the skin of patient 28. In the example of FIG. 1, system 10 comprises six electrodes, of which electrodes 38a, 38b, and 38c are coupled to the front (e.g., chest) of patient 28, and electrodes 38d, 38e, and 38f are coupled to the back of patient 28. As shown in FIG. 1, the electrodes are arranged in pairs as follows: electrodes 38a and 38d are facing one another at the right side of patient 28, electrodes 38c and 38f are facing one another at the left side of patient 28, and electrodes 38b and 38e are facing one another at the upper part of the chest and back of patient 28.

In other embodiments, system 20 may comprise any suitable number of electrodes, coupled to the patient skin in any suitable arrangement.

In some embodiments, electrodes 38a-38f are typically connected, via a cable 37, to a processor 41 of system 20, which is configured to receive from electrodes 38a-38f electrical signals indicative of the measured impedance, and, based on the received signals, to estimate the position of catheter tip 40 within heart 26 using techniques described herein.

In some embodiments, electrodes 38a-38f are typically used for navigating catheter 21 within the body of patient 28, using the aforementioned impedance-based ACL system and tracking techniques, such as those described, for example, in U.S. Pat. No. 8,456,182 and US Patent Application Publication 2015/0141798, whose disclosures are incorporated herein by reference.

In some embodiments, the ACL system is configured to estimate the position of catheter tip 40 responsively to the different impedances measured between the electrode(s) coupled to catheter tip 40, and each of electrodes 38a-38f.

In some embodiments, processor 41 is configured to estimate the position of catheter tip 40 in heart 26, and to display on a display 27 of console 24, a marker (not shown) overlaid on an anatomical image 42 (or a synthetic model) of heart 26. Physician 30 may use the marker, e.g., for navigating catheter tip 40 into ostium 51.

In some embodiments, once distal end 22a of shaft 22 has reached heart 26, physician 30 retracts sheath 23 and further manipulates shaft 22 to navigate catheter tip 40 to ostium 51 of the pulmonary vein, or to any other target location of heart 26.

In some embodiments, while catheter tip 40 is placed in contact with the tissue, physician 30 may control system 20 for acquiring unipolar intra-cardiac electrical signals from the target tissue of heart 26, and/or for applying unipolar ablation pulses to the target tissue.

In principle, physician 30 may acquire the unipolar signals using a return electrode (also referred to herein as an indifferent electrode or a neutral electrode) patch that is coupled externally to the skin of patient 28. Similarly, physician 30 may use the return electrode for applying the one or more unipolar ablation pulses to the tissue of heart 26. The return electrode may be selected from any of electrodes 38a-38f, or from an electrode patch used in any other configuration of electrodes, such as Wilson Central Terminal (WCT). Based on this configuration, physician 30 may control RF generator 45 to apply pulses of RF electric currents to be passed between electrodes 77 of catheter tip 40 and the selected indifferent electrode patch that is coupled externally to the skin of patient 28. In this configuration, however, the large distance between electrodes 77 (in contact with the target tissue of heart 26) and the selected indifferent electrode may (i) add noise to the unipolar signals acquired from the tissue, and/or (ii) increased impedance to the unipolar signals applied to the tissue during the RF ablation procedure.

Techniques to overcome the added noise and/or the increased impedance are described in detail in FIG. 2 below.

Processor 41 is typically a general-purpose computer, with suitable front end and (a) ECG interface circuits 44 for receiving ECG signals from electrodes 38, and (b) an electrical interface circuit 52 for receiving signals from catheter 21, as well as for applying RF energy treatment via catheter 21 in a left atrium of heart 26 and for controlling the other components of system 20. Processor 41 typically comprises a software in a memory 48 of system 20 that is programmed to carry out the functions described herein. The software may be downloaded to the computer in electronic form, over a network, for example, or it may, alternatively or additionally, be provided and/or stored on non-transitory tangible media, such as magnetic, optical, or electronic memory.

This particular configuration of system 20 is shown by way of example, in order to illustrate certain problems that are addressed by embodiments of the present invention and to demonstrate the application of these embodiments in enhancing the performance of such an ablation system. Embodiments of the present invention, however, are by no means limited to this specific sort of example system, and the principles described herein may similarly be applied to other sorts of ablation systems.

In other embodiments, instead of balloon 66, catheter tip 40 may have any other suitable component, such as an expandable basket or any other suitable type of an expandable frame.

### Performing Unipolar Ablation Using a Return Electrode Integrated in Ablation Balloon Catheter

FIG. 2 is a schematic, pictorial illustration of catheter tip 40, in accordance with an embodiment of the invention.

In some embodiments, balloon 66 or any other expandable frame that is coupled to catheter tip 40 of catheter 21, is typically in a collapsed position when physician 30 moves catheter tip 40 to the target location, and is configured to be expanded at the target location.

In the context of the present disclosure, the expandable frame has a collapsed position and an expanded position, and specifically, balloon 66 has a collapsed position and an inflated position, which corresponds to the expanded position of the expandable frame. Note that the description below refers to balloon 66, but the techniques described below may be applied, mutatis mutandis, to any catheter having other sorts of expandable frames, such as but not limited to a basket catheter.

In some embodiments, balloon 66 has a diameter of about 12 mm, or any other suitable diameter, and comprising electrodes 77 disposed on the surface of balloon 66. In some embodiments, when placed in contact with tissue of heart 26, electrodes 77 are configured to sense intra-cardiac electrical signals from the tissue. In the example of FIG. 2, balloon 66 has multiple electrodes 77 so as to obtain high-resolution mapping of the electrical signals in tissue. In the present example, catheter tip 40 is configured to acquire from the tissue unipolar intra-cardiac electrical signals.

In the context of the present disclosure and in the claims, the terms “about” or “approximately” for any numerical values or ranges indicate a suitable dimensional tolerance that allows the part or collection of components to function for its intended purpose as described herein.

In some embodiments, electrodes 77 are further configured to apply to the tissue, one or more unipolar RF ablation pulses received from RF generator 45 and controlled by processor 41 and/or by physician 30, as described in FIG. 1 above. In some embodiments, by using one electrode 77 in contact with the tissue, physician 30 may obtain high-resolution ablation (e.g., form a narrow lesion) at a desired locations of the ablated tissue.

In some embodiments, physician 30 may determine the depth of lesion by controlling, inter alia, the energy and duration of RF ablation pulses applied to the tissue at the target location.

In some embodiments, catheter tip 40 comprises electrode 55, which is coupled to shaft 22 at a selected proximity to balloon 66, and is configured to serve as a return electrode. Note that when balloon 66 is in an inflated position, one or more electrodes 77 are placed in contact with the target tissue (e.g., of ostium 51), but electrode 55 is placed in contact with the blood pool of heart 26 but not directly with the target tissue. Therefore, electrode 55 can serve as a return electrode for sensing unipolar signals from the target tissue and/or for applying unipolar ablation pulses to the target tissue.

In some embodiments, catheter tip 40 may comprise electrode 50, which is disposed within the internal volume of balloon 66 and is therefore shown in FIG. 2 as a dashed element, at a position that is not placed in contact with the target tissue. For example, electrode 50 may be coupled to shaft 22 along an axis 74 of catheter tip 40. In this configuration, when balloon 66 is in an inflated position, one or more electrodes 77 are placed in contact with the target tissue. Electrode 50, however, is within the internal volume of balloon 66, and therefore, is not placed in contact with the target tissue, but with another tissue (e.g., the blood pool of heart 26) or with a saline solution used for inflating balloon 66. Note that both the blood and the saline solution are electrically conductive, and therefore, are configured to conduct the sensed signals and/or the applied pulses described above, so that electrode 50, as well as electrode 55, may serve as a return or common electrode as described above. Moreover, in case electrode 50 or 55 makes contact with the target tissue together with one or more electrodes 77, the aforementioned sensed signals and or applied pulses may be bipolar, e.g., between electrodes 77 and 50 or between electrodes 77 and 55, which is undesired and, in some cases, may cause damage to heart 26.

In some embodiments, balloon 66 may comprise a flexible substrate, such as but not limited to a flexible printed circuit board (PCB), having printed electrical interconnections. In the present example, the electrical interconnections comprise electrical traces 76, which are parallel to axis 74 of catheter tip 40, and electrical traces 78, which are orthogonal to axis 74. The flexible PCB is wrapped around the surface of balloon 66, so that electrical traces 76 and 78 are configured to conduct electrical signals and/or RF ablation pulses between electrode 77 and console 24.

The configuration of balloon 66 is provided by way of example, in order to illustrate certain problems that are addressed by embodiments of the present invention and to demonstrate the application of these embodiments in enhancing the performance of a catheter tip of such an ablation catheter. Embodiments of the present invention, however, are by no means limited to this specific sort of example catheter tip, and the principles described herein may similarly be applied to other sorts of ablation catheters.

In other embodiments, instead of balloon 66, catheter tip 40 may have any other suitable component, such as a basket-type distal end having an expandable frame, which is coupled to shaft 22 of catheter 21. In such embodiments, electrodes 77 may be coupled to splines of the expandable frame of the basket, and electrode 50 may be coupled to shaft 22 within the internal volume of the expandable frame. Additionally or alternatively, electrode 55 may be coupled to shaft 22, e.g., at the position shown in FIG. 2. Note that electrodes 50 and 55 are configured to serve as return or common electrodes for the electrical signals sensed by electrodes 77, therefore, catheter tip 40 may comprise only one of electrodes 50 and 55. In this configuration, electrode 50 or electrode 55 is in contact with the blood pool of heart 26 and is not placed in contact with the target tissue of heart 26.

### Producing Ablation Balloon Catheter Having a Return Electrode

FIG. 3 is a flow chart that schematically illustrates a method for producing catheter tip 40, in accordance with an embodiment of the invention. The method begins at a substrate receiving step 100 with receiving the flexible PCB having electrical interconnections, such as electrical traces 76 and 78. At an ablation electrodes disposing step 102, one or more ablation electrode(s) 77 are disposed on the flexible PCB and connected to electrical traces 76 and 78. In the context of the present disclosure and in the claims, the term “dispose” refers to “form” or “produce” (electrodes 77 in the PCB, e.g., using any suitable PCB production process) or to “couple” or “attach” (electrodes 77 to the PCB, using any suitable coupling technique, such as but not limited to soldering).

At a return electrode coupling step 104, one or more return electrodes 50 and 55, but typically one return electrode is sufficient, is coupled to catheter tip 40 at distal end 22a of catheter 21. Note that the position of electrodes 50 and 55 is provided by way of example, so as to shown that, when balloon 66 or any other suitable type expandable frame is in an inflated or expanded position, one or more electrodes 77 are placed in contact with the target tissue, but neither electrode 50 nor electrode 55 is placed in contact with the target tissue. In other embodiments, a return or common electrode may be coupled to or produced in the expandable frame (e.g., balloon 66 or the aforementioned basket) at any suitable position, so that the return or common electrode does not make contact with the target tissue, when electrodes 77 are placed in contact with the target tissue for sensing the intra-cardiac electrical signals or for applying the one or more ablation pulses to the target tissue, as described in detail in FIGS. 1 and 2 above.

At a catheter tip assembling step 106 that concludes the method, the flexible PCB is wrapped around and coupled to balloon 66 (e.g., using bonding or soldering), and balloon 66 is coupled to distal end 22a of shaft 22 so as to complete the formation of catheter tip 40. Note that in case of using the aforementioned basket instead of balloon 66, step 106 may comprise coupling the basket to distal end 22a of shaft 22 so as to complete the production of catheter tip 40.

The configuration of catheter tip 40 and the production method thereof are simplified and described for the sake of conceptual clarity so as to show the key features of the disclosed invention.

Although the embodiments described herein mainly address unipolar sensing and ablation of cardiac tissue, the methods and systems described herein can also be used in other applications, such as in performing unipolar sensing and/or ablation in any other tissue of the patient body.

It will thus be appreciated that the embodiments described above are cited by way of example, and that the present invention is not limited to what has been particularly shown and described hereinabove. Rather, the scope of the present invention includes both combinations and sub-combinations of the various features described hereinabove, as well as variations and modifications thereof which would occur to persons skilled in the art upon reading the foregoing description and which are not disclosed in the prior art. Documents incorporated by reference in the present patent application are to be considered an integral part of the application except that to the extent any terms are defined in these incorporated documents in a manner that conflicts with the definitions made explicitly or implicitly in the present specification, only the definitions in the present specification should be considered.

What is claimed is:

1. A catheter, comprising:
a catheter shaft;
an expandable frame configured to be inserted into an organ of a patient, and extending distally from the catheter shaft, an internal volume defined within the expandable frame;
one or more first electrodes disposed on the expandable frame at one or more first positions to contact a target tissue of the organ, and are configured to perform one or both of: (i) sensing one or more electrical signals from the target tissue, and (ii) applying one or more ablation pulses to the target tissue; and
a second electrode disposed on the catheter shaft at a second position out of contact with the target tissue while the one or more first electrodes contact the target tissue, the second electrode configured to serve as a return or common electrode for the electrical signals, the second electrode being smaller than the one or more first electrodes combined in surface area.

2. The catheter according to claim 1, comprising an ablation power source, which is electrically connected to the catheter and is configured to apply one or more unipolar ablation pulses to the one or more first electrodes.

3. The catheter according to claim 1, the organ comprises a patient heart, and the one or more electrical signals comprise one or more unipolar intra-cardiac electrical signals sensed from the heart.

4. The catheter according to claim 1, the organ comprises a patient heart, and the one or more ablation pulses comprise one or more unipolar radiofrequency (RF) ablation pulses applied to the heart.

5. The catheter according to claim 1, the expandable frame comprises an inflatable balloon.

6. The catheter according to claim 1, the expandable frame comprises an expandable basket.

7. The catheter according to claim 1, the one or more first electrodes and the second electrode being configured to be inside the organ, the organ being a heart, the one or more first electrodes and the second electrode configured so that when the expandable frame is in an expanded position, the one or more first electrodes are placed in contact with the target tissue and the second electrode is placed in contact with a blood pool of the heart and not in contact with the target tissue.

8. The catheter according to claim 7, the second electrode configured as a return electrode configured to sense unipolar signals from the target tissue.

9. The catheter according to claim 7, the second electrode configured as a return electrode configured to apply unipolar ablation pulses to the target tissue.

10. The catheter according to claim 1, the second electrode disposed within the internal volume of the expandible frame.

11. The catheter according to claim 1, the second electrode disposed outside the internal volume of the expandible frame.

12. A method for producing a catheter, the method comprising:
receiving an expandable frame configured to insert into an organ of a patient;
disposing on the expandable frame, at one or more first positions, one or more first electrodes configured to be placed in contact with a target tissue of the organ; and
disposing a second electrode, configured as a return or common electrode for the electrical signals on the catheter shaft, at a second position that is not in contact with the target tissue while the one or more first electrodes contact the target tissue, the second electrode being smaller than the one or more first electrodes combined in surface area.

13. The method according to claim 12, comprising electrically connecting an ablation power source configured to apply one or more unipolar ablation pulses to the one or more first electrodes.

14. The method according to claim 12, disposing the one or more first electrodes comprises coupling the one or more first electrodes to the expandable frame.

15. The method according to claim 12, disposing the one or more first electrodes comprises producing the one or more first electrodes on the expandable frame.

16. The method according to claim 12, receiving the expandable frame comprises receiving an inflatable balloon.

17. The method according to claim 12, receiving the expandable frame comprises receiving an expandable basket.

18. The method according to claim 12, comprising:
disposing the second electrode out of the internal volume of the expandable frame; and
disposing a third electrode configured as an additional return electrode for the electrical signals at a third position that is out of contact with the target tissue while the one or more first electrodes contact the target tissue, disposing the third electrode on a catheter shaft in the internal volume of the expandable frame.

19. The method according to claim 12, the one or more first electrodes and the second electrode being configured to be inside the organ, the organ being a heart.

20. The method according to claim 12, when expandable frame is in an expanded position, the one or more first electrodes are configured to be placed in contact with the target tissue, the second electrode is configured to be placed in contact with a blood pool of the heart and not in contact with the target tissue.
